(19) United States
(12) Patent Application Publication
(10) Pub. No.: US 2007/0134711 A1
(43) Pub. Date: Jun. 14, 2007

(54) Compositions of eleutherosides capable of modulating protein expresion

(76) Inventors: Vincent R. Bonagura, Englewood Cliff, NJ (US); James DeVoti, Fort Salonga, NY (US); Herman W. Lance, Birmingham, AL (US); John M. Mayhall JR., Birmingham, AL (US)

Correspondence Address: BRADLEY ARANT ROSE & WHITE, LLP;INTELLECTUAL PROPERTY DEPARTMENT-NWJ, 1819 FIFTH AVENUE NORTH, BIRMINGHAM, AL, 35203-2104, US

(21) Appl. No.: 11/652,275

(22) Filed: Jan. 11, 2007

Related U.S. Application Data
(63) Continuation of application No. 10/357,913, filed on Feb. 4, 2003, now abandoned.
(60) Provisional application No. 60/360,788, filed on Mar. 1, 2002.
(60) Provisional application No. 60/354,397, filed on Feb. 4, 2002.

(51) Int. Cl. C12Q 1/70; C12Q 1/68

(52) U.S. Cl. 435/6; 435/5

(57) ABSTRACT

Vaccines containing adjuvant comprising eleutherosides and related compounds are shown to be useful for the prevention of viral infections, bacterial infections and parasitic infections. The adjuvant compounds have been shown to modulate the expression of a wide variety or proteins involved in the immune response and inflammatory response. Exemplary eleutherosides and related compounds include eleutheroside A, eleutheroside B, eleutheroside C, eleutheroside D, eleutheroside E, eleutheroside F, and eleutheroside G, coniferylaldehyde, caffeic acid ethyl ester, chlorogenic acid, sinapinalcohol, isofraxidin, syringaresinol and 6,8-dimethoxy-7-hydroxycoumarin.

This disclosure claims the benefit of and priority to U.S. Ser. No. 60/360,788, filed Mar. 1, 2002 and is a continuation of U.S. Ser. No. 10/357,913, filed on Feb. 4, 2003.

### FIELD OF THE DISCLOSURE

This disclosure relates to the use of eleutherosides as adjuncts for vaccines in subjects, including humans, for the prevention and/or treatment of microbial infections, viral infections and/or cancer. The disclosure also relates to the use of eleutherosides to affect the expression of proteins associated with immune responses to microbial infection, viral infection and/or cancer.

### BACKGROUND

Despite advances made in the prevention and treatment of microbial infections and viral infections pathogenic organisms continue to pose problems for medical science. Currently, diseases such as malaria, AIDS, tuberculosis, influenza, and yellow fever pose real threats to the lives of millions of people worldwide. Though treatments for some infections do exist, these are often expensive and rarely provide a cure for the ailment. As such, our best line of defense against pathogenic organisms is prevention through the use of vaccines.

The use of vaccines had its modern day start with the use of Vaccinia virus (referred to as “cowpox”) for the prevention of smallpox. This action led to the eventual eradication of smallpox in the 1970's. Over the last 50 years there has been increased efforts to develop vaccines against a wide variety of microbes and viruses, including, but not limited to, Micobacterium tuberculosis (BCG vaccine), Polio (Salk and Sabin vaccines), Yellow Fever, Varacella Zoster Virus, Hepatitis B Virus, and Human Immunodeficiency Virus (HIV). Unfortunately, the majority of these vaccines have limited effect, often requiring multiple vaccinations to “boost” the immunological responses to sufficient levels to provide protection. Additionally, the vaccines themselves can have undesirable effects, such as, but not limited to, fever, nausea, vomiting, pain, swelling, and rashes. Therefore, there is a very real need for improved vaccines.

The need for new vaccines particularly applies to HIV infection, for which there is no vaccine approved for use in humans. A vaccine capable of being applied to combat HIV is critically needed as the pandemic associated with HIV infection has reached frequencies as high as 1 in every 5 people in some countries of Southeast Asia (for example, Thailand) and 1 in 3 persons in Africa. The ability of HIV to infect a cell is highly dependent on the viruses ability to bind its receptor CD4 as well as a co-receptor. For all strains of HIV the main co-receptor is the β-chemokine receptor, CCR5, which belongs to the chemokine receptor family whose members include CCR2, CCR2a, CCR2b, and CCR6. Additional co-receptors have been identified and include CXCR4 and CCR2b. By decreasing the expression of a co-receptor for HIV, the ability of the virus to maintain a chronic infection or initially infect an individual is dramatically decreased.

One method of improving vaccines is through the use of adjuncts. Adjunct s are agents administered with the vaccine that accomplish a related, but independent function. The present disclosure provides adjuncts that may induce or prevent the release of cytokines, chemokines, or other agents, thereby modulating the immune response to a given antigenic stimulus. Such functions may result in increased efficacy by stimulating T cells through increased IL-2 production, increased antibody production via IL-6, or induction of apoptosis through proteins such as heat shock protein (HSP). Adjuncts may also decrease side-effects by blocking the production of action of inflammatory cytokines. Because of the above properties, adjuvants may allow vaccines to contain decreased amounts of immunogen and still produce the desired immunoprotective response. In addition, the adjuvant may allow the vaccine to be tolerated better by the patient.

### SUMMARY OF THE DISCLOSURE

The present disclosure describes the use of adjuncts for vaccines, wherein the adjuncts comprise eleutherosides or other components of an Eleutherococcus senticosus extract. The method may be practiced on subjects, including, but not limited to, humans. The extract may modulate (i.e., increase/stimulate or diminish/inhibit) the expression of molecules. Molecules whose expression are increased as a result of eleutherosides or other components from an Eleutherococcus senticosus extract include, but are not limited to, IL-10; heat-shock protein (HSP) family members 70, 70b, 70-2, 40, and 90; c-Fos; junB; heat shock transcription factor-4; and human activating transcription factor (ATF-3). Molecules whose expression is decreased as a result of eleutherosides or other components from an Eleutherococcus senticosus extract include, but are not limited to include fibroblast growth factor receptor; human lymphoid transcription factor; MHC class II HLA-DR2-DW12; CD14 differentiation antigen; human T lymphocyte-specific protein tyrosine kinase (Lck), platelet derived endothelial growth factor; chemokine receptor (CCR) family members CCR2, CCR2a, CCR2b, CCR5, and CCR6; and tumor necrosis factor (TNF). The eleutherosides or other components from an Eleutherococcus senticosus extract may be provided through the use of the following extracts: CM-4, PCM-4, and/or EB-1. Other extracts may be used as well.

Inhibition may occur via direct methods such as directly binding factors already present in the subject or indirectly by preventing synthesis. Stimulation may occur by direct stimulation of cells producing the molecules or by stimulating cells that release factors whose actions result in stimulating another cell to produce the molecule. The adjunct compositions of the disclosure can be components of anti-viral vaccines. The anti-viral vaccines can include vaccines against HIV, Varacella Zoster virus, Herpes Simplex virus-1, Herpes Simplex V=virus-2, Cytomegliavirus, Epstein-Barn virus, Yellow Fever virus, Ebola virus, Influenza virus, Polio virus, Variola virus, rhinovirus, Measles, Mumps, Rubella, Hepatitis A virus, Hepatitis B virus, Hepatitis C virus, Hepatitis D virus, Dengue, Rotavirus, Rabies Japanese B encephalitis, Human Papillomavirus, St. Louis encephalitis virus, Human T lymphocyte virus-1, and Respiratory Syncytial virus. The adjunct can reduce the amount of the immunogen necessary to provide a prophylactic or therapeutic effect. This would result in a safer vaccine, such as the case for live-attenuated or low-dosage vaccines where the immunogen can still cause unwanted effects.

The adjunct compositions of the disclosure can be components of anti-bacterial vaccines. The anti-bacterial vaccines can include, but are not limited to, vaccines against Mycobacterium sp. (such as M. tuberculosis), Vibrio sp. (such as V. Cholera), Mvcobacterium (such as M. leprae), Clostridium sp. (such as C. tetani), Bacilis sp. (such as B. anthracis), enterotoxic Escherichia sp. (such as E. coli), Hemophilus sp. (such as H. influenzae B), Helobacter sp. (such as H. pylori), Pertussis sp., Heliobacter sp., Diptheria sp., Shigella sp., Meningococcus sp., Pneumococcus sp., Streptococcus sp., shilgellosis, tetanus and typhoid. The adjunct can reduce the amount of the immunogen necessary to provide a prophylactic or therapeutic effect. This would result in a safer vaccine, such as for live-attenuated or low-dosage vaccines where the immunogen can still cause unwanted effects.

The adjunct compositions of the disclosure can be components of anti-parasitic vaccines. The anti-parasitic vaccines can include vaccines against Malaria (Plasmodium sp.), schistomiasis (Schistosoma sp.), and leishmaniasis (Leishmania sp.). The adjunct can reduce the amount of the immunogen necessary to provide a prophylactic or therapeutic effect. This would result in a safer vaccine such as for live-attenuated or low-dosage vaccines where the immunogen can still cause unwanted effects.

### BRIEF DESCRIPTION OF THE DRAWINGS

FIG. 1 shows the effect of Eleutherococcus senticosus maxim extract on TNF production in the HL-60 cell line.

### DETAILED DESCRIPTION OF THE DISCLOSURE

Definitions and Nomenclature:

It is to be understood that this disclosure is not limited to specific synthetic methods, specific pharmaceutical carriers, or to particular pharmaceutical formulations or administration regimens, as such may, of course, vary. It is also to be understood that the terminology used herein is for the purpose of describing particular embodiments only and is not intended to be limiting.

It must be noted that, as used in the specification and the appended claims, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like.

The adjunct composition of the disclosure can contain eleutherosides. In addition to or in the alternative to eleutherosides, the adjunction composition can contain compounds such as coniferylaldehyde, caffeic acid ethyl ester, chlorogenic acid, sinapinalcohol, isofraxidin, 6,8-dimethoxy-7-hydroxycoumarin, and syringaresinol. For a description of these compounds and how to prepare them, see U.S. Ser. No. 10/142,412 (filed May 9, 2002), which is incorporated herein in its entirety by this reference. Examples of an adjunct composition of the disclosure include, but are not limited to, CM-4, PCM-4, and EB-I. Herein, the term “eleutherosides” refers to any one of or mixture of eleutherosidic compounds that may be combined with a suitable carrier to be discussed below. Eleutherosides of the disclosure can included one or more of eleutherosides A, B, C, D, E, F, or G. For a description of eleutherosides and how to prepare them, see U.S. Ser. No. 10/142,412 (filed May 9, 2002) which is incorporated herein in its entirety by this reference, for its teaching of the structure and synthesis of eleutherosides and related compounds.

By the term “therapeutic amount” of a compound as provided herein is meant a nontoxic but sufficient amount of the compound to provide the desired activity. As will be pointed out below, the exact amount required will vary from subject to subject, depending on the species, age, and general condition of the subject, the severity of disease that is being treated, the particular compound used, its mode of administration, and the like. Thus, it is possible the specify an exact “therapeutic amount.” However, an appropriate therapeutic amount may be determined by one of ordinary skill in the art using only routine experimentation.

For the purposes of this disclosure, the tern “inhibiting” as it relates to levels of factors such as, but not limited to, TNF; human lymphoid transcription factor; CD 14 differentiation antigen; MHC class II HLA-DR2-DW12; fibroblast growth factor receptor; Lek; platelet derived endothelial growth factor; CCR family members CCR2, CCR2a, CCR2b, CCR5, and CCR6 in subjects and cell lines refers to both the prevention of production of said factors in the respective subjects or cell lines and also to prevention of the activity of any those factors which are already present in the subject or cell line. For example, inhibition of TNF can refer to the ability of eleutherosides or mixtures containing eleutherosides such as, but not limited to, CM-4, PCM-4, or EB-1, to prevent cellular production of TNF as in Example I with the HL-60 cell line or as in Example II with a animal subject. For the teaching of how to prepare and administer CM-4, see U.S. Ser. No. 10/255,915 (filed Sep. 26, 2002), which is incorporated herein in its entirety, by this reference. In some embodiments of the disclosure the EB-1 fraction of PCM-4 is used. For the process of obtaining the EB-1 fraction of PCM-4, please see the section that describes extract preparation, which describes EB-I as the alcohol base extract of the Eleutherococcus senticosits maxim rhizome. In addition, as shown in Example III below, inhibition of TNF can also refer to the ability of eleutherosides to reduce the deleterious effects of existing TNF levels. Thus, in Example III, CM-4 was able to significantly reduce reverse transcriptase activity (i.e. viral replication) in OM-10.1 cells despite the presence of levels of TNF sufficient to stimulate viral replication.

By the term “inhibiting amount” of a composition as provided herein is meant a nontoxic but sufficient amount of the inhibiting composition to provide the desired activity. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “inhibiting amount.” However, an appropriate “inhibiting amount” may be determined by one of ordinary skill in the art using only routine experimentation.

For the purposes of this disclosure, the term “inducing” as it relates to levels of factors such as IL-10; heat-shock protein (HSP) family members 70, 70b, 70-2, 40, and 90; c-Fos; junB; heat shock transcription factor-4; and human activating transcription factor (ATF-3) in subjects and cell lines refers to both the initiation of production of said factors in the respective subjects or cell lines and also to induction of the activity of any those factors which already is present in the subject or cell line.

As with “inhibiting amount” the term “inducing amount” of a composition as provided herein is meant a nontoxic but sufficient amount of the inducing composition to provide the desired activity. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “inducing amount.”However, an appropriate inducing amount may be determined by one of ordinary skill in the art using only routine experimentation.

Eleutherosides and other components of the adjunct composition can exist in pharmaceutically acceptable carriers. A “pharmaceutically acceptable” material is one that is not biologically or otherwise undesirable, i.e., a material that may be administered to an individual along with the selected composition without causing any undesirable biological effects or interacting in a deleterious manner with any of the other components of the pharmaceutical composition in which it is contained. Examples of an eleutheroside in a suitable carrier, include, but are not limited to CM-4, PCM-4 and EB-1, which are described below.

One particular embodiment of this disclosure involves the method of preventing or decreasing HIV infection by decreasing a protein necessary for HIV fusion and entry into T cells. The method involves administering a composition comprising eleutherosides in a sufficient amount as to decrease the expression of chemokine receptor (CCR) family members. One family member, CCR5, is well known in the art to be a necessary co-receptor for HIV entry into a CD4+ T cell for all known strains of HIV. Family member CCR2b has also been shown to be involved as a co-receptor for HIV entry into a cell. Example II below shows via microarray analysis that administration of eleutherosides in a pharmaceutically acceptable carrier such as, but not limited to, EB-1 can decrease the expression levels of these proteins.

Chemokine receptors are also involved in inflamatory responses. An embodiment of this disclosure involves the method of treating inflammatory conditions such as Rheumatoid Arthritis. The method involves administering a composition comprising eleutherosides in a sufficient amount as to decrease the expression of chemokine receptor (CCR) family members. CCR2a is known in the art to play a role in a variety of inflammation (sic) models. Example II below shows via microarray analysis that administration of eleutherosides such as, but not limited to, EB-1 can decrease the expression levels of these proteins.

Another embodiment of this disclosure involves the method of decreasing the debilitating effects associated with late stage HIV infection and AIDS. The method involves administering a composition comprising eleutherosides in a sufficient amount as to alter the expression of TNF-∀ and IL-10. The relationship between TNF-∀ and IL-10 can play a major role in the wasting syndrome associated with the late stages of HIV infection and AIDS progression. Wasting syndrome is also a common problem among the elderly population. Wasting syndrome is defined as unintended and progressive weight loss often accompanied by weakness, fever, nutritional deficiencies and chronic diarrhea. The syndrome, also known as cachexia, can diminish the quality of life, exacerbate illness and increase the risk of death for people with HIV. Researchers have found that increased levels of immune-signaling molecules (cytokines) such as TNF-α are associated with wasting, specifically HIV-induced wasting. Drugs that block TNF-α are predicted to have a role in the treatment of this condition. Example II below shows via microarray analysis that administration of eleutherosides such as, but not limited to, EB-1 can decrease the expression levels TNF-∀ and increase the expression of IL-10

It must also be understood that the term ‘subjects’ as used herein is meant to include animals including humans. In addition, the dilutions of Eleutherococcus extracts are meant to be dilutions in cell culture medium or other neutral carrier. For example, a 1:300 dilution includes 1 part Eleutherococcus extract to 300 parts cell culture medium. By “extract” it is meant an active composition obtained from Eleutherococcus senticosus maxim wherein the Eleutherococcus root is a primary component in the form of a solid, liquid, paste or the like.

Extract Preparation:

Extracts can be obtained by various methods. One extract, CM-4, is a protic extract of the Eleutherococcus root in an ethanol-water extract. Another composition, of the disclosure is the EB-1 fraction of PCM-4, which is an extract of the Eletherococcus senticosus rhizome, in an ethanol base extract.

The preferred process for preparing Eleutherococcus senticosus maxim extract is as follows. Eleutherococcus senticosus maxim root is extracted, shaken free of dirt and sand, and washed with cold flowing water. The root is then dried by baking for one hour at 80° C. After baking, the roots are allowed to continue drying in a vacuum drier at 60° C. or in a warm, dry and well-ventilated location. Then roots are selected based on the following criteria: 1) the root is whole or in small parts, the bark on the outside is a yellow-brown color and internally the root is white, 2) the outside bark of the root is wrinkled, indicating that it has dried properly, 3) breaking the root open reveals a white, slightly fibrous, strongly scented aromatic characteristic odor, and 4) the taste is of the root is spicy and slightly bitter.

The selected roots are then ground into a medium-coarse powder. This powder is mixed with a protic solvent such as 40% ethanol (60% water) by volume for the extraction (1 kg powder per liter of 40% ethanol) and the mixture is heated (Pharmacopeia of USSR, page 522). The extraction can be accomplished with other protic solutions, such as water or alcohol-water solutions, for example 10-100% ethanol solutions with water.

The resulting extract is a darkish brown fluid of a specific color, is transparent, and is bitter. This extract has a dry residue of not less than 6% (Pharmacopeia, supra, p. 253), and an alcohol content of not less than 33% (Pharmacopeia, supra, p. 813).

Administration:

The eleutheroside of the disclosure may be conveniently formulated into various pharmaceutical compositions composed of eleutheroside extract in association with a pharmaceutically acceptable carrier. See, e.g., Remington The Science and Practice of Pharmacy, 20th edition (Lippincott Williams and Wilkins, Baltimore, Md.) discloses typical carriers and conventional methods of preparing pharmaceutical compositions that may be used in conjunction with the preparation of formulations.

Depending on the intended mode of administration, the pharmaceutical compositions may be in the form of solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. The compositions will include, as noted above, an effective amount of eleutheroside in combination with a pharmaceutically acceptable carrier and, in addition, may include other medicinal agents, pharmaceutical agents, carriers, adjuvants, diluents, etc.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., eleutheroside as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; for example, see Remington The Science and Practice of Pharmacy, referenced above.

For oral administration, fine powders or granules may contain diluting, dispersing, and/or surface active agents, and may be presented in water or in a syrup, in capsules or sachets in the dry state, or in a nonaqueous solution or suspension wherein suspending agents may be included, in tablets wherein binders and lubricants may be included, or in a suspension in water or a syrup. Where desirable or necessary, flavoring, preserving, suspending, thickening, or emulsifying agents may be included. Tablets and granules are preferred oral administration forms, and these may be coated.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system, such that a constant level of dosage is maintained. See, e.g., U.S. Pat. No. 3,710,795, which is incorporated by reference herein. A specific example of administration of eleutherosides and E. Senticosus extracts to patients please see U.S. Pat. No. 10/114,340 (filed Apr. 2, 2002), which is incorporated by reference for its teaching with regard to administration and dosages.

### EXPERIMENTAL

The following examples are put forth so as to provide those of ordinary skill in the art with a complete disclosure and description of how the eleutherosides used in the methods claimed herein are made and evaluated, and are not intended to limit the scope of what the inventors regard as their disclosure. Efforts have been made to ensure accuracy with respect to numbers (e.g., amounts, temperature, etc.) but some errors and deviations should be accounted for.

### Example I

### HL-60

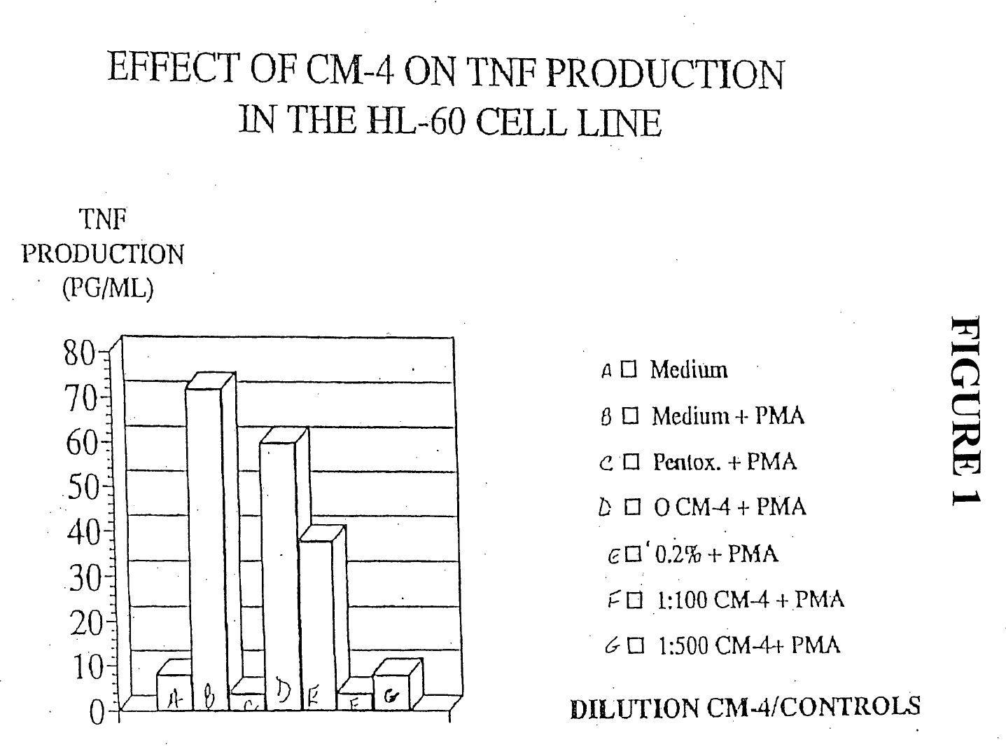

HL-60 cells are a human leukemia cell line known to produce high levels of TNF in response to Phorbol myristate acetate (PMA). In order to assay the effects of eleutherosides on HL-60, HL-60 cells (2×10⁶) in supplemented RPMI-1640 medium were incubated four hours in the presence of various dilutions of extract—in this case CM-4. PMA was added and the incubation continued at 37° C. in 95% air 5% CO₂. TNF production was determined by ELISA. Controls were cells alone, cells with 33% ethanol, and cells with pentoxaphyllin. The results are detailed in FIG. 1.

The results in FIG. 1 show the effect of CM-4 on TNF production in the HL60 cell line. Specifically, HL-60 showed a marked decrease in TNF production upon treatment with 1:100 dilution CM-4 and 1:500 dilution CM-4. At the 1:100 dilution TNF production was less than 5 pg/ml whereas the control registered about 70 pg/ml. At 1:500, CM-4 limited TNF production to less than 10 pg/ml. See FIG. 1. In addition, addition of pentoxaphyllin resulted in very little TNF production (<5 pg/ml). Therefore, CM-4 in dilutions from about 1:100 to 1:500 proved to be highly effective in inhibiting TNF production in HL-60 cells.

### Example II

### Immune Modulation by Adjunct Composition

The adjunct composition was used to stimulate cells derived from human subjects. In order to study the effect of eleutherosides on the gene expression of a subject, cDNA from subjects were applied to a micro-array chip analysis. Briefly, peripheral blood mononuclear cells (PBMC) are removed from whole blood of normal healthy donors by centrifuging the blood on a buffy coat layer. The cells are then treated with CM-4 or EB-1 and incubated for 5 hours at 37C in a 5% CO, incubator. Next RNA is prepared using an RNeasy mini kit (Qiagen; Valencia, Calif.) or similar product and following the manufacturer's instructions. cDNA synthesis was conducting following instructions supplied by GibcoBRL (Carlsbad, Calif.) for use with SuperScript Choice system, labeling, and subsequent processing are conducted following the instructions supplied by Affymetrix (Santa Clara, Calif.) for use with the Affymetrix micro-array.

The results of the micro-array analysis on an Affymetrix HG-U95A microarray chip reveal that cells from subjects treated with eleutherosides showed over a 300-fold increase in the expression levels of HSP. TNF was found to have diminished expression levels that were approximately 300-fold lower in treated individuals than in non-treated individuals. A change greater than 10-fold was considered significant. The results also indicate changes in the level of expression associated with EB-1 administration. These results, as shown in appendices 1-3, reveal that following EB-1 administration there is an increase in the expression levels of IL-10; heat-shock protein (HSP) family members 70, 70b, 70-2, 40, and 90; c-Fos; junB; heat shock transcription factor-4; and human activating transcription factor (ATF-3). Furthermore, the results indicate that there is a decrease in the expression levels of human lymphoid transcription factor; MHC class II HLA-DR2-DW12; CD14 differentiation antigen; fibroblast growth factor receptor; Lck; platelet derived endothelial growth factor; chemokine receptor (CCR) family members CCR2; CCR2a; CCR2b, CCR5, and CCR6; and tumor necrosis factor (TNF).

Additionally, TNF expression has been inhibited by the EB-1 fraction of PCM-4 in human peripheral blood mononuclear cells transduced with the MHC class I proteins E6 and E7 from human papillomavirus 6 and 11. These same cells show enhanced IL10 production. To study the expression of the transduced peripheral blood mononuclear cells, microarray analysis was performed in the manner as described above.

### Example III

### OM 10.1

OM 10.1 is a derivative of the HL-60 cell line that is latently infected with HIV-1. In response to TNF-∀, HIV is induced to produce viral particles.

The OM 10.1 cell line, stock cultures, obtained from the AIDS Research and Reference Reagent Program, NIAID, were subjected to 2 passages in the presence of 5:g/ml AZT prior to use in order to decrease background p24 and reverse transcriptase (RT) levels due to superinfection. The cells thus prepared demonstrated increased p24 and RT levels when subjected to stimulation with TNF-∀ with a concomitant decrease in CD4 expression as measured by FACS analysis following incubation with OK T4 antibody (Becton-Dickenson).

For experimental use, OM 10.1 cells were harvested from a stock culture and washed by centrifugation with Dulbecco's phosphate-buffered saline. The cells were resuspended in cell culture medium (RPMI 1640 medium) containing 10% fetal bovine serum (heat-inactivated at 56° C. for 30 minutes, 100 IU/ml penicillin, 100 μg/ml streptomycin, 20 μg/ml gentamicin, 8 μg/ml tylosin, and 25 mM HEPES). The cultures were maintained in a humidified atmosphere of 5% CO₂ in disposable plastic cell culture ware. The cells were seeded at 800,000 cells/2 ml/well in six well plates and an equal volume of drug dilution in cell culture medium was added. Following 1 hour of incubation, 20 U/ml of TNF-∀ was added to appropriate wells. Culture fluids were harvested after 48 hours of incubation for determination of RT activity and p24 content. Standard methods were used in the RT assays and in the ELISA assays for HIV p24 antigen. The results of this assay are summarized in Table 1.

Table 1 shows the effect of varying concentrations of CM-4 on HIV replication by inhibition of TNF-∀ in latently infected cells. The data indicate that at dilutions of from about 1:300 to 1:600, the CM-4 extract is not toxic to the subject cells. Furthermore, the, data indicate that reverse transcriptase activity is significantly inhibited in OM-10.1 cells by the addition of the CM-4 extract when TNF-∀ is present. Therefore, the CM-4 extract was effective in preventing stimulation of virus production/replication when administered at non-cytotoxic levels.

TABLE 1
Evaluation of Eleutherosides for Inhibition of the
Stimulation by TNF-∀ of Active Virus Production
in OM 10.1 Cells Latently Infected with HIV

| TNF-α | CM-4^{A} | CPM (RT)^{B} | Viability^{C} | Cytotoxicity | % S^{D} | % I^{E} |
|---|---|---|---|---|---|---|
| − | — | 10804 | — | — | — | — |
| − | 1:200 | 5157 | 70% | Toxic | — | — |
| − | 1:300 | 13324 | 92% | Non-toxic | 2 | — |
| − | 1:400 | 12231 | 92% | Non-toxic | 1 | — |
| − | 1:600 | 20027 | 96% | Non-toxic | 7 | — |
| + | — | 148547 | — | — | — | — |
| + | 1:200 | 9125 | 62% | Toxic | — | — |
| + | 1:300 | 31770 | 87% | Non-toxic | — | 85 |
| + | 1:400 | 58825 | 84% | Non-toxic | — | 65 |
| + | 1:600 | 91779 | 95% | Non-toxic | — | 42 |

^{A}Refers to the final concentration (dilution).
^{B}RT is used as the abbreviation for reverse transcriptase activity which is measured by standard methods. CPM is counts per minute of radioactivity.
^{C}Viability was measured by trypan blue exclusion hemacytometer cell counts.
^{D}% S is the stimulation of reverse transcriptase activity seen in relation to that produced by TNF-α. The eleutheroside preparation did not produce significant stimulation of virus production.
^{E}% I is the inhibition of TNF-a stimulated RT activity seen in the presence of eleutherosides. Eleutherosides were effective in preventing the stimulation of virus at non-cytotoxic concentrations.

What is claimed is:

1. A method of modulating the level of a protein in a subject in need of such modulating, said method comprising administering to the subject a therapeutic amount of a composition comprising at least one of the compounds selected from the group consisting of: eleutheroside, coniferylaldehyde, caffeic acid ethyl ester, chlorogenic acid, sinapinalcohol, isofraxidin, syringaresinol and 6,8-dimethoxy-7-hydroxycoumarin.

2. The method of claim 1 where the protein is at least one of the proteins selected from the group consisting of: interleukin (IL)-10, heat shock protein (HSP)-70b, HSP-70-2, HSP-40, HSP-90, heat shock transcription factor-4, c-Fos, junB, activating transcription factor (ATF)-3, tumor necrosis factor (TNF)-α, human lymphoid transcription factor, CD14 differentiation antigen, major histocompatibility class (MHC) class II HLA-DR2-DW12., Lymphocyte Specific Protein Tyrosine Kinase (Lck), fibroblast growth factor (FGF) receptor, platelet derived endothelial growth factor (PDEGF), C-C chemokine receptor type (CCR) 2, CCR2a, CCR2b, CCR5 and CCR6.

3. The method of claim 1 where said modulation is an increase and said protein being is selected from the group consisting of: IL-10, HSP-70b, HSP-70-2, HSP-40, HSP-90, heat shock transcription factor-4, c-Fos, junB and ATF-3.

4. The method of claim 1 where said modulation is a decrease and said protein being is selected from the group consisting of: TNF-α, human lymphoid transcription factor, CD14 differentiation antigen, MHC class II HLA-DR2-DW12., Lck, fibroblast growth factor receptor, platelet derived endothelial growth factor, CCR2, CCR2a. CCR2b, CCR5 and CCR6.

5. The method of claim 1 where said subject is a human.

6. The method of claim 1 where said eleutheroside is selected from the group of compounds selected from the group consisting of eleutheroside A, eleutheroside B, eleutheroside C, eleutheroside D, eleutheroside E, eleutheroside F, and eleutheroside G.

7. The method of claim 1 wherein the composition further comprises a pharmaceutically acceptable carrier.

8. The method of claim 1 where said composition is used as an adjunct.

9. A method of increasing the level of a protein in a subject in need of such increasing, said method comprising administering to the subject a therapeutic amount of a composition comprising at least one of the compounds selected from the group consisting of: eleutheroside, coniferylaldehyde, caffeic acid ethyl ester, chlorogenic acid, sinapinalcohol, isofraxidin, syringaresinol and 6,8-dimethoxy-7-hydroxycoumarin.

10. The method of claim 7 where the protein is at least one of the proteins selected from the group consisting of: IL-10, HSP-70b, HSP-70-2, HSP-40, HSP-90, heat shock transcription factor-4, c-Fos, junB and ATF-3.

11. The method of claim 7 where said subject is a human.

12. The method of claim 7 where said eleutheroside is selected from the group of compounds selected from the group consisting of eleutheroside A, eleutheroside B, eleutheroside C, eleutheroside D, eleutheroside E, eleutheroside F, and eleutheroside G.

13. The method of claim 7 wherein the composition further comprises a pharmaceutically acceptable carrier.

14. The method of claim 7 where said composition is used as an adjunct.

15. A method of decreasing the level of a protein in a subject in need of such decreasing, said method comprising administering to the subject a therapeutic amount of a composition comprising at least one of the compounds selected from the group consisting of: eleutheroside, coniferylaldehyde, caffeic acid ethyl ester, chlorogenic acid, sinapinalcohol, isofraxidin, syringaresinol and 6,8-dimethoxy-7-hydroxycoumarin.

16. The method of claim 15 where the protein is at least one of the proteins selected from the group consisting of: TNF-α, human lymphoid transcription factor, CD14 differentiation antigen, MHC class II HLA-DR2-DW12., Lck, fibroblast growth factor receptor, platelet derived endothelial growth factor, CCR2, CCR2a. CCR2b, CCR5 and CCR6.

17. The method of claim 15 where said subject is a human.

18. The method of claim 15 where said eleutheroside is selected from the group of compounds selected from the group consisting of eleutheroside A, eleutheroside B, eleutheroside C, eleutheroside D, eleutheroside E, eleutheroside F, and eleutheroside G.

19. The method of claim 15 wherein the composition further comprises a pharmaceutically acceptable carrier.

20. The method of claim 15 where said composition is used as an adjunct.
